(19) United States
(12) Patent Application Publication
(10) Pub. No.: US 2017/0231927 A1
(43) Pub. Date: Aug. 17, 2017

(54) PHARMACEUTICAL COMPOSITIONS OF MEMANTINE

(71) Applicant: TEVA PHARMACEUTICAL INDUSTRIES LTD., Petach Tikva (IL)

(72) Inventors: ELENA KAGAN, REHOVOT (IL); NITZAN SHAHAR, KIBBUTZ TEL-YITZHAK (IL); ELINA HARONSKY, ROSH HA'AIN (IL); GREGG R. DEROSA, SOUDERTON, PA (US)

(21) Appl. No.: 15/583,317

(22) Filed: May 1, 2017

Related U.S. Application Data
(63) Continuation of application No. 13/969,161, filed on Aug. 16, 2013, now abandoned.
(60) Provisional application No. 61/683,875, filed on Aug. 16, 2012.
(60) Provisional application No. 61/833,348, filed on Jun. 10, 2013.

(51) Int. Cl. A61K 31/13; A61K 9/48; A61K 9/20; A61K 9/00

(52) U.S. Cl. CPC A61K 31/13; A61K 9/0053; A61K 9/4866; A61K 9/2054; A61K 9/4858; A61K 9/2013; A61K 9/2031; A61K 9/485; A61K 9/2009

(57) ABSTRACT

The present invention relates to oral dosage forms comprising Memantine or a pharmaceutically acceptable salt thereof, pharmaceutical formulations comprising the oral dosage forms, and methods for treating mild, moderate or severe Alzheimer's dementia, or neuropathic pain comprising the oral dosage forms and formulations.

### CROSS-REFERENCE TO RELATED APPLICATIONS

The present application is a continuation of U.S. Ser. No. 13/969,161, filed Aug. 16, 2013, which claims the benefit of priority to U.S. Provisional Application Nos. 61/683,875, filed Aug. 16, 2012, and 61/833,348, filed Jun. 10, 2013, the entire contents of each of which are herein incorporated by reference.

### FIELD OF THE INVENTION

The present invention relates to oral dosage forms comprising Memantine or a pharmaceutically acceptable salt thereof, pharmaceutical formulations comprising the oral dosage forms, and methods for treating comprising the oral dosage forms and formulations.

### BACKGROUND OF THE INVENTION

Memantine is reportedly an orally active NMDA receptor antagonist. The reported IUPAC name for memantine hydrochloride is 3,5-dimethyladamantan-1-amine hydrochloride. Memantine HCl has the structure,

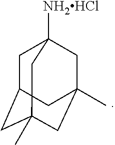

Memantine HCl is currently marketed by Forest in the form of film coated tablets under the trade name NAMENDA®. In Europe, memantine hydrochloride is marketed by Merz (AXURA®) and Lundbeck (EBIXA®). Memantine 10 mg twice daily is the FDA-approved regimen for the treatment of moderate to severe Alzheimer's disease.

NAMENDA XR™ (memantine hydrochloride) was approved by the U.S. Food and Drug Administration for the treatment of moderate to severe dementia of the Alzheimer's type. NAMENDA XR is a 28 mg once-daily extended-release formulation of NAMENDA.

Immediate release pharmaceutical formulations comprising memantine are described in various publications including, inter alia, US 2006/198884 and US 2010/028427.

Modified release pharmaceutical formulations comprising memantine are described in various publications including, inter alia, U.S. Pat. No. 5,382,601, U.S. Pat. No. 6,194,000, U.S. Pat. No. 7,619,007, U.S. Pat. No. 8,039,009 U.S. Pat. No. 8,168,209, US 2007/065512 and US 2010/266684.

Alzheimer's disease is a progressive, degenerative brain disease. As the disease progresses, patients and caregivers face increasing problems with medication adherence. Given Alzheimer's relentlessly progressive nature, newer and more effective therapies for Alzheimer's disease are needed.

### SUMMARY OF THE INVENTION

The present invention provides modified release solid oral dosage forms comprising a distinguishably high amount of memantine or a pharmaceutically acceptable salt thereof while avoiding undesirable side effects, particularly CNS side effects. The high doses in the solid oral dosage forms of the present invention are adapted to achieve therapeutically effective steady-state concentrations at a greater interval between dosing than presently employed, while maintaining safety requirements and improving patient compliance.

The present invention provides modified release solid oral dosage forms comprising a therapeutically effective amount of memantine or a pharmaceutically acceptable salt thereof, and at least one pharmaceutically acceptable rate controlling excipient.

In particular, the present invention provides a modified release solid oral dosage form comprising a therapeutically effective amount of memantine or a pharmaceutically acceptable salt thereof, and at least one pharmaceutically acceptable rate controlling excipient, wherein the solid oral dosage form is adapted for administering with an interval between doses of 5 days or above 5 days to a patient in a need thereof, and wherein the solid oral dosage form:
(a) provides an in vivo plasma profile at steady state comprising a Cₘₐₓ of about 160 ng/ml or less, a Cₘᵢₙ of more than about 30 ng/ml, and an AUCₜₐᵤ of more than about 14,000 ng h/ml, and/or
(b) has a dissolution profile of: not more than 45% at 24 hours, not more than 70% at 48 hours, and not more than 80% at 55 hours.

The modified release solid oral dosage forms of the present invention provide an in vivo plasma profile at steady state comprising Cₘₐₓ of about 160 ng/ml or less, Cₘᵢₙ of more than about 30 ng/ml, AUCₜₐᵤ of more than about 14,000 ng h/ml, and Tₘₐₓ of at least about 36 hours.

The amount of memantine or a pharmaceutically acceptable salt thereof in the modified release solid oral dosage forms of the present invention is preferably up to 200 mg. Preferably, the amount is at least 112 mg, at least 140 mg, at least 160 mg, at least 170 mg, at least 180 mg, or at least about 190 mg of memantine or a pharmaceutically acceptable salt thereof. More preferably, the amount is from about 112 mg to about 200 mg, from about 140 mg to about 200 mg, from about 160, 170, 180 or 190 mg to about 200 mg. In another preferred embodiment, the amount is from about 160 mg to about 190 mg or from about 170 mg to about 190 mg.

The present invention further provides modified release solid oral dosage forms comprising at least about 112 mg, e.g. about 140 mg of memantine or a pharmaceutically acceptable salt thereof, and at least one pharmaceutically acceptable rate controlling excipient, wherein the solid oral dosage form is adapted for administering once weekly to a patient in a need thereof, and wherein the solid oral dosage form provides an in vivo plasma profile at steady state comprising a Cₘₐₓ of about 160 ng/ml or less.

In a preferred dosage form of the invention, the memantine or pharmaceutically acceptable salt thereof is present in both immediate release form and extended release form, and may be in the form of coated beads. According to this aspect, of the invention, the dosage form comprises: (i) an immediate release component comprising immediate release memantine or a pharmaceutically acceptable salt thereof, and (ii) an extended release component comprising extended release memantine or a pharmaceutically acceptable salt thereof. Preferably, the immediate release component (i) is in the form of beads comprising immediate release memantine or a pharmaceutically acceptable salt thereof, and the extended release component (ii) is in the form beads comprising extended release memantine or a pharmaceutically acceptable salt thereof, wherein beads comprise an extended-release coating containing at least one rate controlling excipient, i.e. the formulation contains two populations (IR and ER) of memantine or a pharmaceutically acceptable salt thereof. These dosage forms may be in the form of capsules or tablets.

Alternatively, another preferred dosage form of the present invention comprises memantine or pharmaceutically acceptable salt thereof, wherein the memantine is provided in a matrix comprising a mucoadhesive agent. In this embodiment, the mucoadhesive agent functions as the rate controlling excipient as well as enabling the dosage form to be retained in the body for an extended period of time. Preferably the dosage forms are monolithic, i.e. do not contain layers, and more preferably, the dosage form is a tablet.

The present invention provides methods for treating mild, moderate or severe Alzheimer's dementia, or neuropathic pain, wherein the method comprises administering a modified release solid oral dosage form or pharmaceutical formulation according to the present invention to a patient in need thereof.

The present invention provides modified release solid oral dosage forms or pharmaceutical formulations according to the present invention for use in the treatment of mild, moderate or severe Alzheimer's dementia, or neuropathic pain.

The present invention provides modified release solid oral dosage forms or pharmaceutical formulations according to the present invention for use in the manufacture of a medicament for treating mild, moderate or severe Alzheimer's dementia, or neuropathic pain.

### BRIEF DESCRIPTION OF THE DRAWINGS

FIG. 1 shows the dissolution profile of a 168 mg dose with 5% IR/95% ER (with 30% polymer) population administered once weekly (QW).

FIG. 2 shows the concentration over time curve following administration of 140 mg of memantine QW with a zero order release rate. The P5 and P95 refer the 5% and 95% confidence limits for a 28 mg QD dose.

FIG. 3 shows the concentration over time curve following administration of 168 mg of memantine QW with 10IR/90(ER 30%) population compared to 28 mg QD. The P5 and P95 refer the 5% and 95% confidence limits for a 28 mg QD dose.

FIG. 4 shows the concentration over time curve following administration of 168 mg of memantine QW with 5IR/95(ER 30%) population compared to 28 mg QD. The P5 and P95 refer the 5% and 95% confidence limits for a 28 mg QD dose.

FIG. 5 shows the concentration over time curve following administration of 196 mg of memantine QW with 10IR/90(ER 30%) population compared to 28 mg QD. The P5 and P95 refer the 5% and 95% confidence limits for a 28 mg QD dose.

FIG. 6 shows the concentration over time curve following administration of 196 mg of memantine QW with 5IR/95(ER 30%) population compared to 28 mg QD. The P5 and P95 refer the 5% and 95% confidence limits for a 28 mg QD dose.

### DETAILED DESCRIPTION OF THE INVENTION

Unless stated otherwise, the following terms and phrases as used herein are intended to have the following meanings.

As used herein, the term “C” refers to the plasma/serum/blood concentration of an active pharmaceutical ingredient, or drug, such as memantine or a pharmaceutically acceptable salt thereof, in a biological sample, such as a patient sample (e.g., blood, plasma, serum, and cerebrospinal fluid). The concentration of the drug in the biological sample may be determined by any standard assay method known in the art. The term C includes such concentrations measurements as the Cₘᵢₙ, Cₘₐₓ, AUC, and Cₛₛ (steady state concentration). Typically the term C refers to the plasma, serum or blood concentration.

As used herein, the term “Cₘₐₓ” refers to the maximum plasma, serum or blood concentration of a drug, such as memantine or a pharmaceutically acceptable salt thereof, in a biological sample reached over one dosing interval at steady state. Accordingly, Cₘₐₓ is the maximum concentration at steady state, Cₘₐₓ₋ₛₛ.

As used herein, the term “Cₘᵢₙ” refers to the minimum plasma, serum or blood concentration of a drug, such as memantine or a pharmaceutically acceptable salt thereof, in a biological sample reached over one dosing interval at steady state.

As used herein, the term “Tₘₐₓ” refers to the time required to reach the maximal plasma, serum or blood concentration (“Cₘₐₓ”) of the drug, such as memantine or a pharmaceutically acceptable salt thereof, in a particular patient sample type.

As used herein, the term “AUC” refers to the area under the plasma, serum or blood concentration versus time curve.

As used herein, the term “AUCₜₐᵤ” refers to the area under the curve for a plasma, serum or blood concentration versus time curve of a drug, such as memantine or a pharmaceutically acceptable salt thereof, reached by a given dose over one dosing interval at steady state. The area under the curve is measured for a time tau at steady state, where tau is the length of the dosing interval. The term AUCₜₐᵤ measures the total exposure at steady state for the dosing interval.

As used herein, the term “immediate release” or “IR” means that the escape or release of a drug, such as memantine or a pharmaceutically acceptable salt thereof, from a dosage form (tablet, capsule, pellet, etc.) occurs immediately or as quickly as possible after administration, usually in a few minutes to hours. The drug is released in a single action and the time of action of the drug is limited.

As used herein, the term “modified release” or “MR” means that the escape or release of a drug, such as memantine or a pharmaceutically acceptable salt thereof, from the dosage form (tablet, capsule, pellet, etc.) has been modified so that the release rate is slower than that in an unmodified or immediate release dosage form. Drug release takes place some time after administration and/or for a prolonged period after administration or to a specific target in the body. Drug release may occur over several hours or over several days in order to maintain a therapeutically effective plasma concentration of the drug. Modified release encompasses delayed release (release at a time other than immediately after administration), extended release (release over a prolonged time period), sustained release (rate of drug release is sustained over a period of time), and controlled release (rate of drug release is controlled to get a particular drug concentration in the body) or a combination thereof.

As used herein, the term “rate controlling excipient” refers to an excipient or a combination of excipients present in such amounts sufficient to control the release rate of the drug in the dosage form, for example to reduce the dose-dependent toxicity of a drug, such as memantine or a pharmaceutically acceptable salt thereof. A rate controlling excipient or a combination thereof controls the rate of drug release from a dosage form.

As used herein, the term “at least one pharmaceutically acceptable rate controlling excipient” refers to the presence of one, two, three, four, or more rate controlling excipients in the dosage form. Preferably, one or two rate controlling excipients are employed.

As used herein, the term “memantine” refers to memantine free base. In certain embodiments, memantine also includes any pharmaceutically acceptable salt, such as the HCl salt. Preferably, in any embodiments of the invention as described herein, the memantine is in the form of its hydrochloride salt. More preferably, in any embodiment of the invention as described herein, reference to the amounts and dosage ranges of memantine in the solid oral dosage forms are to the amounts and dosage ranges of memantine hydrochloride.

As used herein, the term “QW” refers to a dosage form suitable for once weekly administration.

As used herein, the term “once weekly” means administering a dose once every seven days. The interval between administrations for once weekly is six days if administration of a dose occurs on the same day each week.

As used herein, the term “QD” refers to a dosage form suitable for once daily administration.

As used herein, the term “Cav” refers to average concentration during a dosing interval. It can be calculated as (AUC₍₀₋ₜₐᵤ₎)/tau.

As used herein, the term “DFL” refers to degree of fluctuation. It can be calculated as 100*(Cₘₐₓ−Cₘᵢₙ)/Cav.

As used herein, reference to a total weight of a dosage form refers to the total weight of a tablet (excluding any non functional coating such as cosmetic coating), and in the case of a capsule, refers to the total weight of the capsule contents, excluding the weight of the capsule itself.

As used herein, unless otherwise indicated, references to percentages refer to weight percent.

Capsule sizes refer to standard sizes known to those skilled in the art. For example, a capsule size of -00- refers to a capsule with a volume of from about 0.9 ml to about 1 ml, typically about 0.95 ml; a capsule size of -0- refers to a capsule with a volume of from about 0.6 ml to about 0.7 ml, typically about 0.68 ml; and a capsule size of -1- refers to a capsule with a volume of from about 0.4 ml to about 0.6 ml, typically about 0.5 ml.

As used herein, the term “bioavailability” refers to the rate and extent to which an active pharmaceutical ingredient is absorbed from a dosage form and becomes available at the site of action.

The present invention provides modified release solid oral dosage forms comprising a therapeutically effective amount of memantine or a pharmaceutically acceptable salt thereof, and at least one pharmaceutically acceptable rate controlling excipient, wherein the solid oral dosage forms are adapted for administering with an interval between doses of 5 days, or more e.g. 6-8 days, preferably 6, 7 or 8 days and more preferably 6 days, to a patient in a need thereof, and wherein the solid oral dosage forms provide an in vivo plasma profile at steady state comprising a Cₘₐₓ of about 160 ng/ml or less, a Cₘᵢₙ of more than about 30 ng/ml, and an AUCₜₐᵤ of more than about 14,000 ng h/ml. For example, it will be appreciated that an interval of 6 days between doses would enable the drug to be administered weekly to the patient, on the same day of the week.

The present invention further provides modified release solid oral dosage forms comprising at least about 112 mg, e.g., about 140 mg of memantine or a pharmaceutically acceptable salt thereof, and at least one pharmaceutically acceptable rate controlling excipient, wherein the solid oral dosage forms are adapted for administering once weekly to a patient in a need thereof, and wherein the solid oral dosage form provides an in vivo plasma profile at steady state comprising a Cₘₐₓ of about 160 ng/ml or less.

The present invention further provides modified release solid oral dosage forms comprising at least about 112 mg, or at least about 140 mg of memantine or a pharmaceutically acceptable salt thereof, and at least one pharmaceutically acceptable rate controlling excipient, wherein the solid oral dosage form is adapted for administering once weekly, most preferably on the same day every week, to a patient in a need thereof, and wherein the solid oral dosage form provides an in vivo plasma profile at steady state comprising a Cₘₐₓ of about 160 ng/ml or less, a Cₘᵢₙ of more than about 30 ng/ml, Tₘₐₓ of at least about 36 hours, and an AUCₜₐᵤ of more than about 14,000 ng h/ml.

In one embodiment, the present invention provides a modified release solid oral dosage form comprising at least about 112 mg, preferably at least about 140 mg, and more preferably at least about 160 mg of memantine or a pharmaceutically acceptable salt of memantine, and at least one pharmaceutically acceptable rate controlling excipient, wherein the solid oral dosage form provides an in vivo plasma profile at steady state comprising a Cₘₐₓ of about 160 ng/ml or less.

The amount of memantine or a pharmaceutically acceptable salt thereof in the modified release solid oral dosage forms of the present invention is up to 200 mg. Preferably, the amount is of at least 112 mg, at least 140 mg, at least 160 mg, at least 170 mg, at least 180 mg, or at least about 190 mg of memantine or a pharmaceutically acceptable salt thereof. More preferably, the amount is from about 112 mg to about 200 mg, from about 140 mg to about 200 mg, from about 160, 170, 180 or 190 mg to about 200 mg, for example, 168 mg, 176 mg, 182 mg, 188 mg and 196 mg.

A pharmacokinetic parameter or combinations of such parameters indicate the bioavailability of an active pharmaceutical ingredient, such as, memantine or a pharmaceutically acceptable salt thereof. Such pharmacokinetic parameters are known to the person skilled in the art. Examples of such parameters include: T₁₁₂ (half-life), Cₘᵢₙ, Cₘₐₓ, AUC, AUCₜₐᵤ, Tₘₐₓ, and Cₛₛ (steady state concentration).

In preferred embodiments, the modified release solid oral dosage forms of the present invention comprise the following in vivo plasma profile concentrations, at steady state: a Cₘₐₓ of about 145 ng/ml or less or about 135 ng/ml or less, more preferably about 125 ng/ml or less; a Cₘᵢₙ of more than about 30 ng/ml, more preferably greater than about 40 ng/ml, and even more preferably a Cₘᵢₙ of more than about 50 ng/ml.

In a specific embodiment of the present invention, the modified release solid oral dosage forms provide an in vivo plasma profile at steady state comprising an AUCₜₐᵤ of more than about 14,000 ng h/ml, preferably more than about 15,000 ng h/ml, most preferably, more than about 16,000 ng h/ml. Optionally, the modified release solid oral dosage forms provide an in vivo plasma profile at steady state comprising an AUCₜₐᵤ of more than about 17,000 ng h/ml.

The solid oral dosage forms of the present invention comprise the following in vivo plasma profile concentrations at steady state: Cₘₐₓ from about 100 ng/ml to about 170 ng/ml, preferably from about 100 ng/ml to about 140 ng/ml or from about 100 ng/ml to about 130 ng/ml; a Cₘᵢₙ from about 20 ng/ml to about 125 ng/ml, preferably from about 30 ng/ml to about 125 ng/ml or from about 40 ng/ml to about 125 ng/ml or from about 50 ng/ml to about 125 ng/ml; and an AUCₜₐᵤ from about 10,000 ng h/ml to about 25,000 ng h/ml, preferably from about 12,000 ng h/ml to about 25,000 ng h/ml or from about 14,000 ng h/ml to about 25,000 ng h/ml or from about 15,000 ng h/ml to about 25,000 ng h/ml or from about 16,000 ng h/ml to about 25,000 ng h/ml or from about 17,000 ng h/ml to about 25,000 ng h/ml.

The solid oral dosage forms of the present invention solve the problem of providing a unit dose of memantine for low frequency administration i.e., more than 5 day intervals (preferably 6 day intervals, i.e. preferably once weekly that also provides a therapeutically effective amount of memantine and with minimal side effects, preferably not more than the side effects associated with Namenda XR® (28 mg, capsule), most preferably not more than the side effects associated with of Namenda® (10 mg, tablet) when these are administered at the recommended dosages (for example once daily for Namenda XR® 28 mg and twice per day for Namenda® 10 mg). Compositions for low frequency administration, for example, once weekly, require higher amounts of memantine. Compositions with higher amounts of memantine would increase undesirable side effects and toxicity because the memantine would be released and transported into the bodily fluids immediately or over a very short period of time after administration. In addition, a therapeutically effective amount of memantine would not be present in the bodily fluids during the prolonged interval between administrations. In order to achieve a desirable combination of pharmacokinetic parameters, higher dose memantine compositions are formulated to control the release of memantine so that a therapeutically effective amount is available in the bodily fluids.

Currently available formulations for memantine, such as those for NAMENDA® and Namenda XR™ which are intended for daily administration, are not suitable for lower frequency administration, for example, for once weekly administration. For example, these formulations do not contain the required amount of memantine or the sufficiently prolonged release characteristics to enable a longer interval between doses. For example, varying the amount of memantine in such formulations in order to provide an acceptable Cₘᵢₙ to be therapeutically effective, would also increase the Cₘₐₓ to a level that is toxic. Lowering the Cₘₐₓ to non-toxic levels in such formulations would result in a Cₘᵢₙ that are too low to be therapeutically effective throughout the interval between administrations. Even if an acceptable Cₘₐₓ and Cₘᵢₙ can be provided, the total exposure (AUC) to memantine during the interval between administration would be too low so that there would be too little memantine in the systemic circulation to achieve appropriate therapeutic activity.

The dosage forms of the present invention solve the problems of providing a desirable combination of pharmacokinetic parameters by modifying the release of memantine by increasing the dose of memantine and including excipient that control the release of memantine. The rate controlling excipients reduce the rate of release of memantine so that the total amount of memantine is not released all at once, but is prolonged over the interval between administrations.

However, the inventors have found that prolonging the release of memantine creates the problem that too little memantine is released early during the interval between administration. As a result, a therapeutically effective amount is not achieved. By including a portion of the total memantine in immediate release form and another portion in extended release form in accordance with the present invention, the inventors have found that it is possible to achieve a sustained release formulation for low frequency administration whilst enabling a therapeutically effective amount of memantine to be achieved soon after administration. The amount of memantine in the immediate release form is a sufficient amount that does not produce a spike in the plasma concentration versus time curve, that is, does not produce a too high Cₘₐₓ that increases toxicity.

The dosage forms of the present invention as described above may further include one or more mucoadhesives to slow the passage of the dosage form through the body e.g. gastrointestinal tract, so that the dosage form remains in the body sufficiently long for all the memantine to be released in the body.

According to a second aspect of the present invention, a sustained release formulation for low frequency administration can also be produced by formulating memantine as a mucoadhesive dosage form, e.g. as a matrix formulation which releases memantine over an extended period while remaining in the gastrointestinal tract. These dosage forms include at least one mucoadhesive excipient providing the required sustained release characteristics to enable low frequency administration as described above. The mucoadhesive dosage forms according to this aspect of the present invention are preferably monolithic, i.e. do not comprise layers.

Treatments of acute and chronic neurological and neuropsychiatric diseases have the problem of treatment compliance because the patient or caretaker may forget to administer the medication. This is especially applicable to the treatment of Alzheimer's disease. The modified release dosage forms of the present invention have the advantage that they can be administered less often and therefore improves compliance.

Accordingly, the oral dosage forms of the present invention provide advantages over other known oral dosages. For example, the oral dosage forms of the present invention can be administered less frequently yet still provide a therapeutic effective amount of memantine and steady state blood levels. The oral dosage forms of the present invention provide reduced pill burden for patients who resist treatment, increase convenience for caregivers who can only check the patient on a weekly basis leading to greater compliance and less burden on family members. In addition, the oral dosage forms of the present invention can be taken with or without food, and for patients who cannot swallow capsules, the oral dosage forms can be sprinkled on applesauce or other food and the entire contents consumed.

The dosage forms described herein are formulated such that the memantine or a pharmaceutically acceptable salt thereof present in the dosage form has an in vitro dissolution profile that is slower than that for an immediate release (IR) formulation as well as slower than that for the 28 mg of memantine extended release NAMENDA XR™ (inactive ingredient are sugar spheres, polyvinylpyrrolidone, hypromellose, talc, polyethylene glycol, ethylcellulose, ammonium hydroxide, oleic acid, and medium chain triglycerides in hard gelatin capsules). The dosage forms of the present invention may contain immediate release, sustained or extended release or delayed release components, or combinations thereof. Preferably, the dosage forms of the present invention comprise a combination of an immediate release component and a sustained or extended release component.

The memantine or a pharmaceutically acceptable salt thereof, in the solid oral dosage forms of the first aspect of the present invention can be provided in a modified release form such as controlled or extended release (ER) form, with an immediate release (IR) component. Thus, the solid oral dosage forms of this aspect of the present invention comprise both an IR (immediate release) component and an ER (extended release) component. In a preferred embodiment, the solid oral dosage form of the invention contains at least 90% of the memantine or a pharmaceutically acceptable salt thereof in an extended release form and the remaining memantine or pharmaceutically acceptable salt thereof in an immediate release form. Another specific embodiment of the invention is a modified release solid oral dosage form, wherein at least 95% of the memantine or a pharmaceutically acceptable salt thereof is in an extended release form and the remaining memantine or pharmaceutically acceptable salt thereof is in an immediate release form.

In one preferred embodiment, the solid oral dosage forms of the present invention comprise about 5% to about 20% of the memantine or a pharmaceutically acceptable salt thereof in immediate release form and about 80% to about 95% of the memantine or a pharmaceutically acceptable salt thereof in extended release form. In a more preferred embodiment, the solid oral dosage forms of the present invention comprise about 5% to about 10%, about 5% to about 15% of the memantine or a pharmaceutically acceptable salt thereof in immediate release form and about 85% to about 95%, about 90% to about 95% of the memantine or a pharmaceutically acceptable salt thereof in extended release form.

The immediate release component of the dosage forms of the present invention can comprise a core coated with layer containing memantine or with a pharmaceutically acceptable salt thereof. The IR component can comprise a sugar sphere, which is coated with a layer containing IR memantine or a pharmaceutically acceptable salt thereof. The IR memantine layer can comprise memantine or a pharmaceutically acceptable salt thereof and a binder (preferably hydroxypropyl methyl cellulose, preferably Methocel E-5 PR). Alternatively, instead of a core (e.g. a sugar sphere) coated with a memantine (and optionally a binder) layer, the IR component can be formed as a core comprising IR memantine, for example, memantine or a pharmaceutically acceptable salt thereof, preferably in combination with a filler (preferably microcrystalline cellulose, e.g. Avicel PH101).

The extended release component of the dosage forms of the present invention can be prepared by coating the beads forming the immediate release component, as defined above, with a controlled release layer. Preferably the controlled release layer comprises a rate controlling excipient optionally with a plasticizer and/or a pore former. More preferably, the controlled release layer comprises a rate controlling excipient together with a plasticizer. Particularly preferred controlled release layers include: (1) at least one rate controlling polymer (preferably ethyl cellulose, more preferably ethyl cellulose 7 cPS, hydroxypropyl methylcellulose, preferably HPMC 6 cPs, or a combination thereof) and at least one plasticiser (preferably triethyl citrate), and (2) a polyacrylate dispersion (preferably an aqueous dispersion of a neutral copolymer based on ethyl acrylate and methyl methacrylate such as Eudragit NE 30D) and at least one pore former and/or an anti-tacking agent (preferably talc). The talc in the latter formulation, as well as functioning as a pore former, may also function as an anti-tacking agent for the controlled release layer.

The controlled release excipient is preferably present in an amount of about 10 to about 50 wt % relative to the weight of the ER memantine component in the dosage form, more preferably about 15 to about 45 wt % and most preferably about 18 to about 38 wt % relative to the weight of the ER memantine component in the dosage form.

Preferably the weight ratio of control release excipient(s) to plasticizer in the ER memantine component of the dosage form ranges from about 10:1 to about 3:1, more preferably about 8:1 to about 4:1, and most preferably about 6:1 to about 5:1.

The amount of the controlled release layer components (e.g. controlled release polymer and plasticizer) relative to the total weight of the ER memantine component of the dosage form is preferably from about 15 to about 50 wt %, more preferably from about 20 to about 35 wt % and particularly from about 20 to about 30 wt %.

Preferably, the weight ratio of the controlled release excipient to memantine or pharmaceutically acceptable salt thereof in the ER memantine component of the dosage form is about 1:1 to about 1:5, more preferably about 1:1 to about 1:4, and particularly about 1:1 to about 1:3, especially about 1:1.5 to about 1:2.5.

Preferably the weight ratio of the controlled release layer components (e.g. controlled release polymer and plasticizer) relative to the remaining components in the ER memantine component of the dosage form is about 1:5 to about 1:1, more preferably about 1:4 to about 1:2, and particularly about 1:3.8 to about 1:3.

Solid oral dosage forms of the present invention comprising both an IR and ER component can be made by mixing an IR component with an ER component. For example, dosage forms having 10% IR and 90% ER, or 5% IR and 95% ER (designated 10IR/90ER and 5IR/95ER) can be made by mixing corresponding portions of IR and ER. The mixture can be filled into a capsule, or compressed into a tablet. Where the dosage form is a tablet, the IR and ER components can be mixed together, optionally with further excipients (e.g. lubricant, filler and optionally a mucoadhesive, as defined herein—preferably magnesium stearate, lactose, starch, and polyethylene oxide) before being compressed into tablets.

Modified release dosage forms can be made by, but not limited to, making pellets of different thicknesses so that the thinnest release the drug first and the thickest last, including a slow dissolving matrix or coating, including a non-dissolving coating around a tablet or capsule with small holes to let the drug out (by diffusion or solvation), controlling release of the drug by diffusion through a coating or matrix or by erosion of the matrix or coating by a process dependent on, for example, a particular condition such as the presence of enzymes or a particular pH. Modified release dosage forms have higher amounts of the drug than the amount present in an unmodified or immediate release dosage form.

The dissolution rate of the dosage forms of the present invention are typically of not more than 35% at 24 hours, not more than 70% at 48 hours, or not more than 80% at 55 hours. In a preferred embodiment, the modified release solid oral dosage forms provide a dissolution rate of not more than 70% at 50 hours, or a dissolution rate of more than 70% at 72 hours or a dissolution rate of more than about 80% at about 96 hours. As used herein, dissolution rates are measured using a USP type 1 (basket) dissolution system at 100 rpm, 900 ml, 0.2% NaCl in 0.1N HCl pH=1.2, at a temperature of 37±0.5° C.

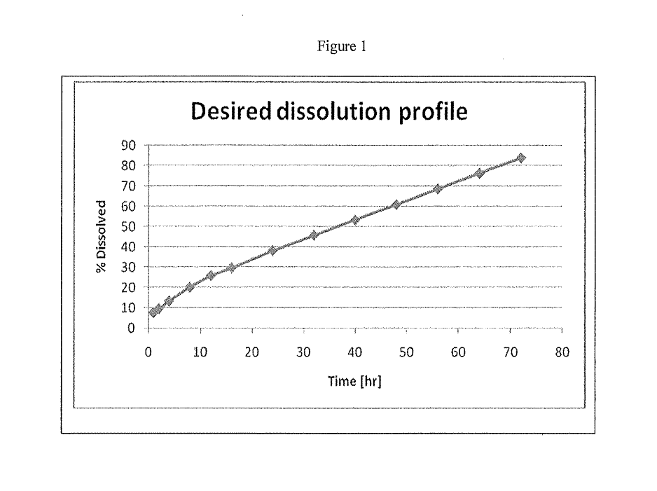

Desirably, a specific dosage form described herein has an in vitro profile that is substantially identical to the dissolution profile shown in FIG. 1. For example, the modified release solid oral dosage forms provide a dissolution rate of:
(i) about 15% to about 25% after 10 hours, about 35% to about 45% after 24 hours and about 55% to about 65% after 48 hours
(ii) preferably about 30% to about 40% after 20 hours, about 50% to about 60% after 40 hours and about 65% to about 80% after 60 hours,
(iii) more preferably about 10% to about 20% after 5 hours, about 40% to about 50% after 30 hours and about 65% to about 75% after 60 hours.

In preferred embodiments, the modified release solid oral dosage forms of the present invention provide in vivo plasma profiles at steady state which are further characterized by a memantine or a pharmaceutically acceptable salt thereof Tₘₐₓ of at least about 36 hours, more preferably a memantine or a pharmaceutically acceptable salt thereof Tₘₐₓ of about 36 to 96 hours. Most preferably, the memantine or a pharmaceutically acceptable salt thereof Tₘₐₓ is of about 48 to 72 hours.

The modified release solid oral dosage forms of the present invention can be adapted for administration with an interval between doses of above 5 days (preferably, of 6 days) to a patient in a need thereof. Preferably, the dosage form is adapted for administration once every 7 days. In a preferred embodiment, the interval between administrations is 5 days, 6 days or 7 days. For example, administration once every 7 days (i.e. with an interval of 6 days) enables administration of the drug on the same day of the week, thus facilitating patient compliance.

The solid oral dosage forms of the present invention include all pharmaceutically acceptable salts of memantine. Preferably, the memantine is in its hydrochloride salt form or in its sulfate salt form. More preferably the memantine is in the form of memantine hydrochloride.

The modified release solid oral dosage form of the present invention is suitable for administration in a one unit dosage form. Oral dosage forms for the purpose of the present invention include capsules, tablets, pellets, granules, powders and combinations thereof. Preferably, oral dosage forms of the present invention are in the form of capsules, tablets, pellets or granules. Particularly preferred are oral dosage forms in the form of capsules or tablets. Optionally, if the dosage form is a capsule, the memantine or a pharmaceutically acceptable salt thereof is provided in the form of coated beads. Typically, if the modified release solid oral dosage form is in a capsule form, the dosage form comprises from about 15% to about 25% by weight of memantine or a pharmaceutically acceptable salt of memantine. Capsule size of the present invention is preferably smaller than -00-, more preferably, -0- or smaller, most preferably -1- or smaller.

Typically, if the modified release solid oral dosage form is in a tablet form, the dosage form comprises from about 10% to about 20% by weight (preferably about 10% to about 18 wt %), or about 15% to about 35% by weight of memantine or a pharmaceutically acceptable salt of memantine. In other embodiments, the modified release solid oral dosage form comprises memantine or the pharmaceutically acceptable salt thereof from about 15% to about 25% by weight of the total dosage form. Typically for dosage forms of the present invention in capsule form, the dosage form comprises from about 20% to about 60%, preferably from about 25% to about 50%, and more preferably from about 28% to about 50%, by weight of memantine or a pharmaceutically acceptable salt of memantine.

The pharmaceutically acceptable rate controlling excipient of the modified release solid oral dosage form according to the first aspect of the present invention can be a polymeric material or combinations of one or more polymeric materials. Preferably, the polymeric material is selected from a group consisting of: polyethylene oxide, ethyl cellulose (e.g., preferably ethylcellulose having a viscosity of about 4 to about 10 cPs, particularly about 7 cPs), hydroxypropyl methylcellulose (HPMC, preferably having a viscosity of about 4 to about 9 cPs, more preferably about 5 to about 8 cPS, and most preferably about 5 to about 7 cPs and particularly about 6 cPs), polyvinyl alcohol (PVA, preferably polyvinyl alcohol 205, 523, 540, 203S, 205S, 523S and 540S), polyvinylpyrrolidone (PVP, preferably Povidone K 12, Povidone K 17, Povidone K 25, Povidone K 30 and Povidone K 90, more preferably Povidone K 25, Povidone K 30 and Povidone K 90). These polymeric materials are preferably combined with a plasticizer to form the controlled (extended) release layer. Suitable plasticizers include those selected from the group consisting of: polyethylene glycol, triethyl citrate, tributyl citrate, glycerine, dibutyl sebacate, triacetin and diethylphthalate. Other suitable rate controlling excipients include polyacrylates, polymethacrylates, ethyl acrylate-methyl methacrylate copolymers (such as Eudragit RS or NE), hydroxypropyl cellulose (HPC, preferably having a viscosity of about 4 to about 9 cPs, more preferably about 5 to about 8 cPS, and most preferably about 5 to about 7 cPs) and a mixture thereof. A particularly preferred rate controlling excipient is a copolymer of ethyl acrylate and methyl methacrylate, for example an aqueous dispersion of a neutral copolymer based on ethyl acrylate and methyl methacrylate (e.g. Eudragit, preferably Eudragit NE30D, which is a 30% aqueous dispersion of the above).

Preferably, the pharmaceutically acceptable rate controlling excipient is selected from a group consisting of: polyethylene oxide, ethyl cellulose (e.g. ethylcellulose having a viscosity of about 4 to about 10 cPs), hydroxypropyl methylcellulose (HPMC), ethyl acrylate-methyl methacrylate copolymers (Eudragit®), and a mixture thereof. In certain preferred embodiments, the rate controlling excipient is a combination of at least two polymeric materials. For example, in some preferred embodiments, the rate controlling excipient is a combination of ethyl cellulose (preferably having a viscosity of about 4 to about 10 cPs, and more preferably about 6 to about 8 cPs), and hydroxylpropylmethyl cellulose (preferably having a viscosity of about 4 to about 9 cPs, more preferably about 5 to about 8 cPS, and most preferably about 5 to about 7 cPs). In some preferred embodiments, the pharmaceutically acceptable rate controlling excipient is a combination of polyethylene oxide and Eudragit®, a combination of ethyl cellulose (having a viscosity of about 4 to about 10 cPs), hydroxypropyl methylcellulose (having a viscosity of about 4 to about 9 cPs) and triethyl citrate, or a combination of polyethylene oxide.

The total amount of the rate controlling excipient, i.e., the polymeric material and other rate controlling excipient, is about 8% to about 60% of the total weight of the dosage form. In a preferred embodiment, the total amount of the rate controlling excipient is from about 8% to about 50% of the total weight of the dosage form, or from about 8% to about 40% of the total weight of the dosage form, or from about 8% to about 30% of the total weight of the dosage form, or from about 8% to about 20% of the total weight of the dosage form, or from about 50% to about 60% of the total weight of the dosage form. Preferable, total amount of the rate controlling excipient is about 19% to about 40% of the total weight of the dosage form, or from about 19% to about 30% of the total weight of the dosage form, or from about 19% to about 25% of the total weight of the dosage form, or from about 30% to about 60% of the total weight of the dosage form, or from about 30% to about 50% of the total weight of the dosage form, or from about 30% to about 40% of the total weight of the dosage form, or from about 40% to about 60% of the total weight of the dosage form, or from about 50% to about 60% of the total weight of the dosage form. Preferably, in the ER memantine component, the memantine or pharmaceutically acceptable salt thereof is present in an amount of about 8 to about 60 wt %, preferably about 10 to about 50 wt %, relative to the ER component. The amount of the rate controlling polymer is about 60% to about 100%, about 70 to about 90%, about 70% to about 80% of the weight of the ER component of the dosage form.

The amount of the rate controlling polymer in the controlled release layer, i.e. not including extragranular part is about 60% to about 100%, about 70 to about 90%, about 70% to about 80% of the total weight of the controlled release layer.

In the ER component of the dosage form, the amount of the plasticizer is about 10% to about 20%, about 12% to about 16%, about 14% to about 15% of the weight of the controlled release layer (e.g. controlled release polymer and plasticizer).

In a preferred embodiment, the ratio of the amount by weight of memantine or a pharmaceutically acceptable salt thereof to the total amount by weight of the rate controlling excipient in the ER component of the dosage form is from about 1:0.3 to about 1:5.0. Preferably, the ratio is from about 1:0.4 to about 1:0.8, or from about 1:2.5 to about 1:4.0.

Preferably, in the dosage forms of the present invention having an immediate release memantine component and an extended release memantine component, the ER memantine component preferably comprises about 30% to about 40%, preferably about 35% by weight of the other parts of the ER memantine component (e.g. core and drug layer). Such dosage forms are designated, for example, as 10IR/90 (ER 30%) or 10IR/90 (ER 35%).

The solid oral dosage forms of the first aspect of the present invention can further comprise one or more mucoadhesives as described below.

As indicated above, a second aspect of the invention provides a solid oral dosage form which comprises memantine or a pharmaceutically acceptable salt thereof in an extended release form in which the memantine or pharmaceutically acceptable salt thereof is formulated with at least one mucoadhesive. Thus, in the dosage forms of this aspect of the present invention, the mucoadhesive functions as both the rate controlling excipient and further enables the dosage form to be retained in the body for an extended time. In particular, the memantine or a pharmaceutically acceptable salt thereof is present in a matrix containing a mucoadhesive. The modified release solid oral dosage forms of the present invention can comprise at least one mucoadhesive with or without an immediate release component or an extended release component as described above. For example, the dosage forms of the present invention can comprise at least one mucoadhesive with only an extended release component, or only immediate release component without controlled release layer.

Alternatively according to a second aspect of the present invention, the memantine may be formulated in a matrix with a mucoadhesive. Preferably the dosage form of this aspect of the present invention is in the form of a monolithic tablet.

Mucoadhesives slow the passage of the dosage form through the body so that the dosage form is inside the body during the interval between administrations so that memantine or a pharmaceutically acceptable salt thereof is released in the body. Mucoadhesives are substances that adhere to a biological tissue for an extended period of time by interfacial forces. The biological tissue is a mucous membrane. Mucoadhesion occur when a mucoadhesive contacts and adheres to a membrane by wetting of the mucoadhesive surface or from the swelling of the mucoadhesive. Further adhesion occurs when the mucoadhesive penetrates into the crevice of the membrane surface or when the chains of the mucoadhesive interact with those of the mucus on the membrane. Suitable mucoadhesive are polymers that are water soluble or water insoluble hydrophilic polymers, polymers that have swellable networks, hydrogels, and polymers with groups that can cross-link with other polymers or with a mucous membrane. For example, mucoadhesives can be polyethylene oxide when used in the matrix with the drug.

In the second aspect of the present invention, where the memantine or pharmaceutically acceptable salt thereof is formulated as a matrix with at least one mucoadhesive, the mucoadhesive may be present from about 20% to about 60%, about 30% to about 60%, about 40% to about 60%, and particularly about 50 to about 60 wt %, of the total weight of the dosage form.

The ratio of the amount by weight of memantine or the pharmaceutically acceptable salt thereof to the amount by weight of the mucoadhesive is from about 1:2 to about 1:4, preferably about 1:4.

In preferred embodiments of the formulations of the present invention wherein the dosage form comprises an IR memantine component and an ER memantine component (wherein the ER component contains memantine coated with an ER layer), polyethylene oxide may be included as an extra-granular layer in the ER component, i.e., not in the core or in the designated controlled release layer. When polyethylene oxide is in the extra-granular layer, it functions as a rate controlling excipient, i.e., the release rate is controlled by both the rate controlling excipients in the controlled release layer and in an extra-granular layer.

The modified release solid oral dosage forms of the present invention may further comprise one or more pharmaceutically acceptable carriers or excipients.

Examples of pharmaceutical acceptable excipients are fillers, binders, glidants, lubricants and bases.

Suitable binders include, for example, cellulose polymers (e.g. hydroxypropylmethyl cellulose, hydroxypropylcellulose, methylcellulose and hydroxyethyl cellulose and METHOCEL™), polyvinylpyrrolidone, polyvinyl alcohol, and mixtures thereof. The amount of binder is about 10% to about 20% of the total weight of the dosage form.

Suitable fillers (diluents) include, for example, microcrystalline cellulose (e.g. Avicel), lactose, sorbitol, dextrose, sucrose, mannitol, dibasic calcium phosphate, starch, and mixtures thereof. The amount of the filler is about 15% to about 60% of the total weight of the dosage form, or about 15% to about 50% of the total weight of the dosage form, or about 15% to about 30% of the total weight of the dosage form.

Suitable glidants include, for example colloidal silicon dioxide, magnesium stearate, talc, sodium stearyl fumarate, magnesium carbonate, starch and mixtures thereof. Preferably the glidant is colloidal silicon dioxide. The amount of the glidant is about 0.1% to about 5% of the total weight of the dosage form, about 0.1% to about 3% of the total weight of the dosage form or about 0.1% to about 1% of the total weight of the dosage form.

Suitable lubricants include, for example, sodium stearyl fumarate, stearic acid, magnesium stearate, calcium stearate, zinc stearate, talc, glyceryl behenate, and mixtures thereof. Preferably, the lubricant is magnesium stearate, talc, and mixtures thereof. The amount of the lubricant is about 0.5% to about 5% of the total weight of the dosage form, or about 2% to about 4% of the total weight of the dosage form.

Suitable bases include sodium carbonate.

Tablets in accordance with this invention can be prepared by conventional mixing, comminution, and tabletting techniques that are well known in the pharmaceutical formulations industry. The modified release tablet, for example, may be obtained by direct compression by punches and dies fitted to a rotary tabletting press, ejection or compression molding, granulation followed by compression, or forming a paste and extruding the paste into a mold or cutting the extrudate into short lengths. Preferably, the process used for preparing tablets is direct compression of the blend.

Compression can be accomplished using conventional equipment. Typically, the blend of active ingredients and excipients is passed through a roller apparatus for compaction. However, other means for compacting the API mixture, e.g., compaction into slugs (or “slugging”), may be used.

To achieve the desired modified release rates, the modified release dosage form may be formulated as a polymeric coating or matrix.

In other embodiments, the present invention provides an oral dosage form comprising a plurality of beads, each bead comprising a core comprising an active ingredient and, in the case of the ER memantine component, a rate controlling excipient layer. The modified release beads in accordance with the present invention may be prepared initially as IR beads, with a core, layer of active ingredient, and a seal (i.e. a cosmetic) coating. Alternatively, the IR beads may be formed of a core containing memantine and optionally a filler. The IR beads may then be coated with a modified release component in the form of a rate controlling excipient dispersion and preferably an additional topcoat of polymer (i.e. a cosmetic coat) for aesthetic, handling or stability purposes. The final dosage form, such as a capsule, may contain a different amount of beads depending on the desired dose of the composition.

The beads or bead mixtures may be used, for example, in suspensions, filled into capsules or compressed into tablets. One or more types of modified release beads can be mixed together and encapsulated.

In one embodiment of the invention, the beads are formulated into capsules with the use of an encapsulation machine.

Alternatively, if the dosage form is to be a tablet, the IR and ER beads may optionally be mixed with further excipients (e.g. lubricant, filler and a mucoadhesive, as defined herein—preferably magnesium stearate, lactose, starch, and polyethylene oxide)

The present invention further provides a method for treating a disorder selected from the group consisting of mild, moderate and severe Alzheimer's dementia, and neuropathic pain, wherein the method comprises administering a therapeutically effective amount of a modified release solid oral dosage form of the invention.

The present invention provides modified release solid oral dosage form or pharmaceutical formulation according to the present invention for use in the treatment of mild, moderate or severe Alzheimer's dementia, or neuropathic pain.

The present invention provides modified release solid oral dosage forms or pharmaceutical formulations according to the present invention for use in the manufacture of a medicament for treating mild, moderate or severe Alzheimer's dementia, or neuropathic pain.

The present invention is illustrated by the following examples, which are not intended to limit the scope of the invention. It will be appreciated that various modifications are within the spirit and scope of the invention.

### EXAMPLES

The pharmacokinetic model used in the examples below can be represented as follows.

PK Model For ER Simulation
Structural Model: 1-compartment PK Model
Inter-Individual Variability: Exponential
CL = TVCL • EXP(ETA1)
V = TVV • EXP(ETA2)
KA = TVKA
ALAG1 = TLAG1 • EXP(ETA3)
K20 = CL/V
S2 = V/1000
CP = A(2)/S2
Residual Variability: Not Estimated

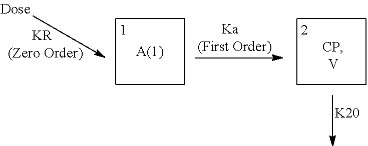

| Parameter | Value | | Parameter | Value |
|---|---|---|---|---|
| Ka | 0.2 | 1/hr | | |
| CL | 9.6 | L/hr | ω²_{CL} | 0.0625 |
| V | 828 | L | ω²_{V} | 0.0625 |
| ALAG1 | 1.5 | hr | ω²_{ALAG1} | 0.0625 |

### Example 1: Exposure Following Administration of 140 Mg of Memantine Once Weekly

Plasma concentration over time was obtained by convoluting actual (in vitro) dissolution data with observed plasma concentration versus time data. The obtained Plasma concentration over time was simulated using a Monte Carlo simulation with a 1-compartment model in NONMEM v 7.1 for 1000 subjects. Phoenix WinNonLin (6.1) was used to perform non-compartmental analysis on the simulated plasma concentration over time. Pharmacokinetic parameters were calculated from the simulated data and are listed below for exposures for once weekly administration of 140 mg of memantine for 7 doses.

| | | Mean | | | | |
|---|---|---|---|---|---|---|
| | Zero order | AUCtau | | | | |
| Dose and | release | (hr * | Cmax | Cmin | Cav | DFL |
| Regimen | rate | ng/mL) | (ng/mL) | (ng/mL) | (ng/mL) | (%) |
| 140 mg QW | 2.04 mg/hr | 14801 | 133.5 | 46.8 | 88.1 | 98 |

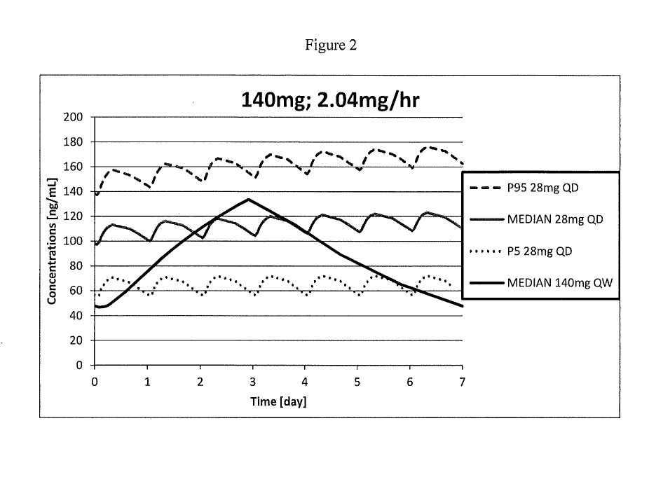

The concentration over time curve following 140 mg of memantine QW administration with a zero order release rate compared to 28 mg QD is shown in FIG. 2.

### Example 2: Exposure Following Administration of Less than 140 Mg of Memantine Once Weekly

Following the procedure in Example 1, pharmacokinetic parameters were calculated for once weekly administration of 28 mg, 56 mg, 84 mg and 112 mg of memantine. The parameters are listed below for exposures to 7 doses.

| | | Mean | | | | |
|---|---|---|---|---|---|---|
| | Zero order | AUCtau | | Cmin | | |
| Dose and | release | (hr * | Cmax | (ng/ | Cav | DFL |
| Regimen | rate | ng/mL) | mL) | (ng/mL) | (ng/mL) | (%) |
| 28 mg QW | 2.04 mg/hr | 2976 | 32.7 | 6.8 | 17.7 | 146 |
| 56 mg QW | 2.04 mg/hr | 5971 | 64.3 | 14.7 | 35.5 | 139 |
| 84 mg QW | 2.04 mg/hr | 8954 | 90.7 | 23.9 | 53.3 | 125 |
| 112 mg QW | 2.04 mg/hr | 11932 | 114.5 | 34.6 | 71.0 | 113 |

### Example 3: Exposure Following Administration of More than 140 Mg of Memantine Once Weekly

Following the procedure in Example 1, pharmacokinetic parameters were calculated for once weekly administration of 168 mg and 196 mg of memantine. The parameters are listed below for exposures to 7 doses.

| | | Mean | | | | |
|---|---|---|---|---|---|---|
| | Zero order | AUCtau | | | | |
| Dose and | release | (hr * | Cmax | Cmin | Cav | DFL |
| Regimen | rate | ng/mL) | (ng/mL) | (ng/mL) | (ng/mL) | (%) |
| 168 mg QW | 2.04 mg/hr | 17704 | 146.0 | 60.9 | 105.4 | 81 |
| 196 mg QW | 2.04 mg/hr | 20302 | 160.3 | 75.1 | 120.8 | 71 |

### Example 4: Exposure Following Administration of 168 Mg of Memantine Once Weekly

Plasma concentration over time was obtained by convoluting actual (in vitro) dissolution data with observed plasma concentration versus time data. The obtained Plasma concentration over time was simulated using a Monte Carlo simulation with a 2-compartment model in NONMEM v 7.1 for 1000 subjects. Phoenix WinNonLin (6.1) was used to perform non-compartmental analysis on the simulated plasma concentration over time. Pharmacokinetic parameters were calculated from the simulated data and are listed below for exposures for once weekly administration of 168 mg of memantine QW for 11 doses.

| | Mean | | | | |
|---|---|---|---|---|---|
| Dose and | AUCtau | Cmax | Cmin | Cav | DFL |
| Regimen | (hr*ng/mL) | (ng/mL) | (ng/mL) | (ng/mL) | (%) |
| 168 mg QW | 14772 | 125.2 | 40.9 | 87.9 | 96 |

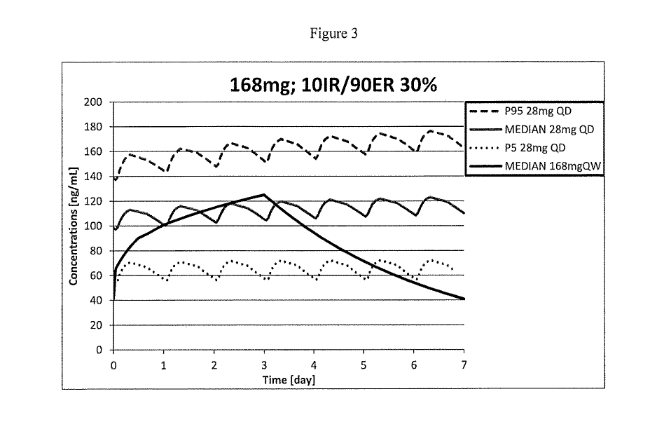

The concentration over time curve following administration of 168 mg of memantine QW with 10 IR/90 (ER 30%) population compared to 28 mg QD is shown in FIG. 3.

### Example 5: Exposure Following Administration of 168 Mg of Memantine Once Weekly

Following the procedure in Example 4, pharmacokinetic parameters were calculated for once weekly administration of 168 mg of memantine with 5IR/95(ER 30%) population. The parameters are listed below for exposures to 11 doses.

| | Mean | | | | |
|---|---|---|---|---|---|
| Dose and | AUCtau | Cmax | Cmin | Cav | DFL |
| Regimen | (hr*ng/mL) | (ng/mL) | (ng/mL) | (ng/mL) | (%) |
| 168 mg QW | 14641 | 126.4 | 41.3 | 87.1 | 98 |

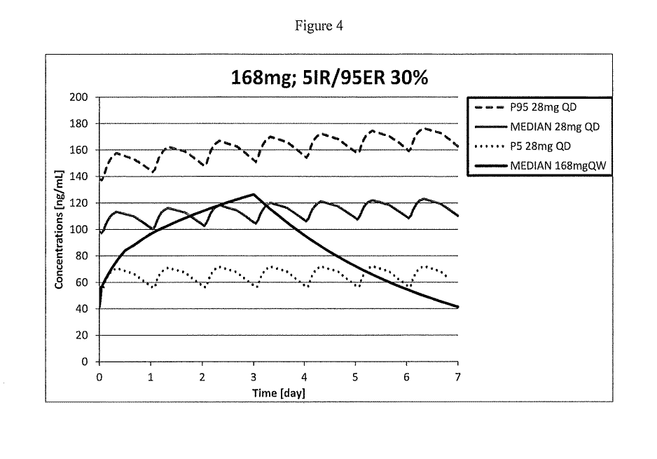

The concentration over time curve following administration of 168 mg of memantine QW with 5IR/95(ER 30%) population compared to 28 mg QD is shown in FIG. 4.

### Example 6: Exposure Following Administration of 196 Mg of Memantine Once Weekly

Following the procedure in Example 4, pharmacokinetic parameters were calculated for once weekly administration of 196 mg of memantine with 10IR/90(ER 30%) population. The parameters are listed below for exposures to 11 doses.

| | Mean | | | | |
|---|---|---|---|---|---|
| Dose and | AUCtau | Cmax | Cmin | Cav | DFL |
| Regimen | (hr*ng/mL) | (ng/mL) | (ng/mL) | (ng/mL) | (%) |
| 196 mg QW | 17234 | 145.9 | 47.7 | 102.6 | 96 |

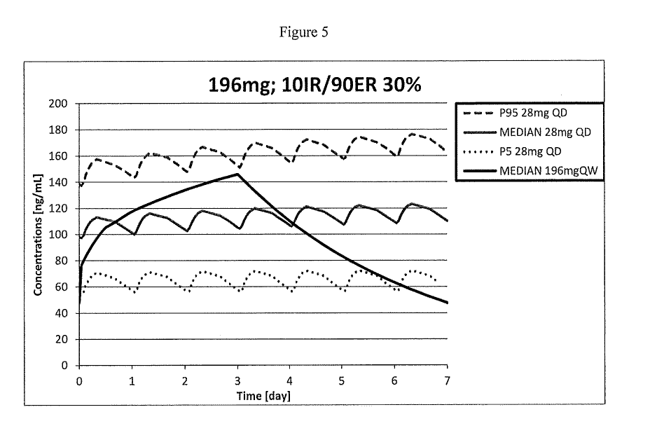

The concentration over time curve following administration of 196 mg of memantine QW with 10IR/90(ER 30%) population compared to 28 mg QD is shown in FIG. 5.

### Example 7: Exposure Following Administration of 196 Mg of Memantine Once Weekly

Following the procedure in Example 4, pharmacokinetic parameters were calculated for once weekly administration of 196 mg of memantine with 5IR/95(ER 30%) population. The parameters are listed below for exposures to for 11 doses.

| | Mean | | | | |
|---|---|---|---|---|---|
| Dose and | AUCtau | Cmax | Cmin | Cav | DFL |
| Regimen | (hr * ng/mL) | (ng/mL) | (ng/mL) | (ng/mL) | (%) |
| 196 mg QW | 17081 | 147.4 | 48.2 | 101.7 | 98 |

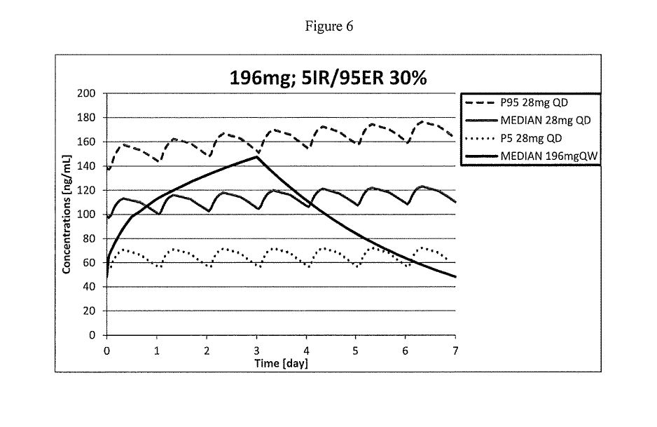

The concentration over time curve following administration of 196 mg of memantine QW with 5IR/95(ER 30%) population compared to 28 mg QD is shown in FIG. 6.

### Example 8: Exposure Following Administration of 176 Mg, 182 Mg and 188 Mg of Memantine Once Weekly

Following the procedure in Example 4, pharmacokinetic parameters were calculated for once weekly administration of 176 mg, 182 mg and 188 mg of memantine with 10IR/90(ER 30%) and 5IR/5(ER 30%) populations. The parameters are listed below for exposures to 11 doses.

### 10IR/90(ER 30%) Population

| | Mean | | | | |
|---|---|---|---|---|---|
| Dose and | AUCtau | Cmax | Cmin | Cav | DFL |
| Regimen | (hr * ng/mL) | (ng/mL) | (ng/mL) | (ng/mL) | (%) |
| 176 mg QW | 15476 | 131.0 | 42.8 | 92.1 | 96 |
| 182 mg QW | 16003 | 135.4 | 44.3 | 95.3 | 96 |
| 188 mg QW | 16531 | 139.9 | 45.8 | 98.4 | 96 |

### 5IR/95(ER 30%) Population

| | Mean | | | | |
|---|---|---|---|---|---|
| Dose and | AUCtau | Cmax | Cmin | Cav | DFL |
| Regimen | (hr * ng/mL) | (ng/mL) | (ng/mL) | (ng/mL) | (%) |
| 176 mg QW | 15338 | 132.4 | 43.3 | 91.3 | 98 |
| 182 mg QW | 15861 | 136.9 | 44.8 | 94.4 | 98 |
| 188 mg QW | 16384 | 141.4 | 46.3 | 97.5 | 98 |

The following Examples 9, 10, 11, 13, 14, 15 and 17 illustrate the extended release component of the dosage forms according to the first aspect of the present invention, i.e. wherein the memantine is coated with an extended or controlled release coating. It will be appreciated that the final dosage form can be prepared by mixing the extended release component with an immediate release component, in the appropriate proportions as required, and either encapsulated, e.g. as illustrated in Example 18, or mixed with tableting excipients and compressed, e.g. as illustrated in Example 19. The immediate release components can be made in the same way as the extended release component, but omitting the controlled release layer.

Examples 12 and 16 illustrate dosage forms according to the second aspect of the present invention, i.e. wherein the memantine is provided in a matrix with a mucoadhesive.

### Example 9: Coated Spheres (Extrusion/Spheronized) Filled into Capsules

Memantine Hydrochloride and microcrystalline cellulose are mixed into a blend with a high shear mixer. The blend is further granulated with gradual addition of water for a few minutes. The resulting wet mass is extruded through a 0.6-1.0 mm screen, and then spheronized in a spheronizer to create spheroids. The spheroids are dried in a Fluid bed Glatt Dyer till the moisture content is less than about 2%, and optionally sieved to provide particles in a selected size range. The resulting spheroids are coated in a Wurster-equipped Fluidized Bed coater (bottom-spray technology) with Control release layer.

Formulation of Beads by Extrusion

| | |
|---|---|
| Component | Weight (mg) |

Core

| | |
|---|---|
| Microcrystalline cellulose (Avicel PH 101) | 210.0 |
| Memantine HCl | 140.0 |

Control Release Layer

| | |
|---|---|
| Ethylcellulose 7 cPs | 73.5 |
| Hydroxypropylmethyl cellulose 6 cPs | 15.75 |
| Triethyl citrate | 15.75 |
| Total weight | 455.0 |

### Example 10: Coated Spheres (DL Coating) Filled into Capsules

Sugar spheres are coated with Drug Layer (DL) containing Memantine HCl and HPMC as a binder dissolved in hydro alcoholic solution in a Wurster-equipped Fluidized Bed coater (bottom-spray technology). The second Control Release coating is also performed in Wurster-equipped Fluidized Bed coater.

Formulation of Beads by DL Coating

| | |
|---|---|
| Component | Weight (mg) |

Core

| | |
|---|---|
| Sugar spheres | 50.0 |

Drug Layer Coating

| | |
|---|---|
| Memantine HCl | 140.0 |
| (METHOCEL E-5 PR.(HYPROMEL USP | 42.0 |

Control Release Layer

| | |
|---|---|
| Ethylcellulose 7 cPs | 48.72 |
| Hydroxypropylmethyl cellulose 6 cPs | 10.44 |
| Triethyl citrate | 10.44 |
| Total weight | 301.6 |

### Example 11: Coated Spheres (DL Coating/Extrusion) Compressed into Tablets

Memantine Hydrochloride and microcrystalline cellulose are mixed into a blend with a high shear mixer. The blend is further granulated with gradual addition of water for a few minutes. The resulting wet mass is extruded through a 0.6-1.0 mm screen, and then spheronized in a spheronizer to create spheroids. The spheroids are dried in a Fluid bed Glatt Dryer till the moisture content is less than about 2%, and optionally sieved to provide particles in a selected size range. Alternatively, Memantine HCl pellets can be obtained by Drug Layer (DL) coating as in Example 10. The resulting spheroids are coated in a Wurster-equipped Fluidized Bed coater (bottom-spray technology) with Control release layer. The extended release coated beads are further mixed with a spray-dried compound consisting of 85% alpha-lactose monohydrate and 15% maize starch dry matter (Starlac™), a non-ionic polyethylene oxide polymer (Polyox WSR-301) and magnesium stearate and then compressed into tablets.

Formulation of Beads compressed into tablets

| | |
|---|---|
| Component | Weight (mg) |

Core

| | |
|---|---|
| Microcrystalline cellulose (Avicel PH 101) | 210.0 |
| Memantine HCl | 140.0 |

Control Release Layer

| | |
|---|---|
| Polyacrylate dispersion (EUDRAGIT NE 30D) | 86.5 |
| Talc extra fine | 13.5 |

Ex-granular

| | |
|---|---|
| Polyethylene oxide (Polyox WSR-301) | 300.0 |
| STARLAC ™ | 300.0 |
| Magnesium stearate | 10.0 |
| Total weight | 1060.0 |

### Example 12: Extended Release Matrix Tablets

Memantine Hydrochloride, non-ionic polyethylene oxide polymer (Polyox WSR-301), microcrystalline cellulose, Lactose, Colloidal silicon dioxide, Sodium Carbonate and Magnesium Stearate are mixed in dry mix and then compressed into tablets. Alternatively, this process can be performed by wet granulation when polyethylene oxide polymer (Polyox WSR-301) Colloidal silicon dioxide, Sodium Carbonate and Magnesium Stearate are added as ex-granular.

Formulation of ER Tablets

| Component | Weight (mg) |
|---|---|
| Memantine HCl | 140.0 |
| Microcrystalline cellulose (Avicel PH 101) | 200.0 |
| Polyethylene oxide (Polyox WSR-301) | 532.5 |
| Lactose | 50.0 |
| Colloidal silicon dioxide | 20.0 |
| Sodium Carbonate | 10.0 |
| Magnesium stearate | 10.0 |
| Total weight | 962.5 |

### Example 13: Beads by Extrusion Spheronization Having Higher Strength

Memantine Hydrochloride and microcrystalline cellulose are mixed into a blend with a high shear mixer. The blend is further granulated with gradual addition of water for a few minutes. The resulting wet mass is extruded through a 0.6-1.0 mm screen, and then spheronized in a spheronizer to create spheroids. The spheroids are dried in a Fluid bed Glatt Dryer till the moisture content is less than about 2%, and optionally sieved to provide particles in a selected size range. The resulting spheroids are coated in a Wurster-equipped Fluidized Bed coater (bottom-spray technology) with Control release layer.

Formulation of Beads by Extrusion

| | |
|---|---|
| Component | Weight (mg) |

Core

| | |
|---|---|
| Microcrystalline cellulose (Avicel PH 101) | 294.0 |
| Memantine HCl | 196.0 |

Control Release Layer

| | |
|---|---|
| Ethylcellulose 7 cPs | 99.2 |
| Hydroxypropylmethyl cellulose 6 cPs | 21.26 |
| Triethyl citrate | 21.26 |
| Total weight | 631.7 |

### Example 14: Beads by DL Technology Having Higher Strength

### For Both Formulation A and Formulation B:

Sugar spheres are coated with Drug Layer (DL) containing Memantine HCl and HPMC as a binder dissolved in hydro alcoholic solution in a Wurster-equipped Fluidized Bed coater (bottom-spray technology). The second Control Release coating is also performed in Wurster-equipped Fluidized Bed coater.

Formulation of Beads by DL Coating (Formulation A)

| | |
|---|---|
| Component | Weight (mg) |

Core

| | |
|---|---|
| Sugar spheres | 57.5 |

Drug Layer Coating

| | |
|---|---|
| Memantine HCl | 161.0 |
| (METHOCEL E-5 PR.(HYPROMEL USP | 57.5 |

Control Release Layer

| | |
|---|---|
| Ethylcellulose 7 cPs | 56.03 |
| Hydroxypropylmethyl cellulose 6 cPs | 12.00 |
| Triethyl citrate | 12.00 |
| Total weight | 346.84 |

Formulation of Beads by DL Coating (Formulation B)

| | |
|---|---|
| Component | Weight (mg) |

Core

| | |
|---|---|
| Sugar spheres | 67.85 |

Drug Layer Coating

| | |
|---|---|
| Memantine HCl | 190.0 |
| (METHOCEL E-5 PR.(HYPROMEL USP | 67.85 |

Control Release Layer

| | |
|---|---|
| Ethylcellulose 7 cPs | 66.11 |
| Hydroxypropylmethyl cellulose 6 cPs | 13.80 |
| Triethyl citrate | 13.80 |
| Total weight | 409.27 |

### Example 15: Compressed Tablets Formulation

Memantine Hydrochloride and microcrystalline cellulose are mixed into a blend with a high shear mixer. The blend is further granulated with gradual addition of water for a few minutes. The resulting wet mass is extruded through a 0.6-1.0 mm screen, and then spheronized in a spheronizer to create spheroids. The spheroids are dried in a Fluid bed Glatt Dryer till the moisture content is less than about 2%, and optionally sieved to provide particles in a selected size range. Alternatively, Memantine HCl pellets can be obtained by Drug Layer (DL) coating as in Example 10. The resulting spheroids are coated in a Wurster-equipped Fluidized Bed coater (bottom-spray technology) with Control release layer. The extended release coated beads are further mixed with a spray-dried compound consisting of 85% alpha-lactose monohydrate and 15% maize starch dry matter (Starlac™), a non-ionic polyethylene oxide polymer (Polyox WSR-301) and magnesium stearate and then compressed into tablets.

Formulation of Beads compressed into tablets

| | |
|---|---|
| Component | Weight (mg) |

Core

| | |
|---|---|
| Microcrystalline cellulose (Avicel PH 101) | 285.6 |
| Memantine HCl | 190.0 |

Control Release Layer

| | |
|---|---|
| Polyacrylate dispersion (EUDRAGIT NE 30D) | 116.8 |
| Talc extra fine | 18.4 |

Ex-granular

| | |
|---|---|
| Polyethylene oxide (Polyox WSR-301) | 408.0 |
| STARLAC ™ | 408.0 |
| Magnesium stearate | 13.6 |
| Total weight | 1442.0 |

### Example 16: Extended Release Matrix Tablets

Memantine Hydrochloride, non-ionic polyethylene oxide polymer (Polyox WSR-301), microcrystalline cellulose, Lactose, Colloidal silicon dioxide, Sodium Carbonate and Magnesium Stearate are mixed in dry mix and then compressed into tablets. Alternatively, this process can be performed by wet granulation when polyethylene oxide polymer (Polyox WSR-301) Colloidal silicon dioxide, Sodium Carbonate and Magnesium Stearate are added as ex-granular.

Formulation of ER Tablets

| Component | Weight (mg) |
|---|---|
| Memantine HCl | 190.0 |
| Microcrystalline cellulose (Avicel PH 101) | 272.0 |
| Polyethylene oxide polymer (Polyox WSR- | 728.3 |
| 301) | |
| Lactose | 68.0 |
| Colloidal silicon dioxide | 27.0 |
| Sodium Carbonate | 13.6 |
| Magnesium stearate | 13.6 |
| Total weight | 1312.5 |

### Example 17

Formulation of Beads by DL Coating

| | |
|---|---|
| Component | Weight (mg) |

Core

| | |
|---|---|
| Sugar spheres | 67.85 |

Drug Layer Coating

| | |
|---|---|
| Memantine HCl | 190.0 |
| (METHOCEL E-5 PR.(HYPROMEL USP | 67.85 |

Control Release Layer

| | |
|---|---|
| Ethylcellulose 7 cPs | 79.8 |
| Hydroxypropylmethyl cellulose 6 cPs | 17.1 |
| Triethyl citrate | 17.1 |
| Total weight | 439.7 |

Sugar spheres are coated with Drug Layer (DL) containing Memantine HCl and HPMC as a binder dissolved in hydro alcoholic solution in a Wurster-equipped Fluidized Bed coater (bottom-spray technology). The second Control Release coating is also performed in Wurster-equipped Fluidized Bed coater. The final coated beads are then encapsulated.

### Example 18: Capsule Dosage Form Containing IR and ER Memantine Components

Formulation of Beads with IR&ER

| | |
|---|---|
| Component | Weight (mg) |

Core

| | |
|---|---|
| Sugar spheres | 67.85 |

Drug Layer Coating

| | |
|---|---|
| Memantine HCl | 190.0 |
| (METHOCEL E-5 PR.(HYPROMEL USP | 67.85 |

IR Portion

| | |
|---|---|
| DL IR Portion | 16.30 |

ER Portion
Control Release Layer

| | |
|---|---|
| Ethylcellulose 7 cPs | 75.8 |
| Hydroxypropylmethyl cellulose 6 cPs | 16.24 |
| Triethyl citrate | 16.24 |
| Total weight | 450.28 |

Sugar spheres are coated with Drug Layer (DL) containing Memantine HCl and HPMC as a binder dissolved in hydro alcoholic solution in a Wurster-equipped Fluidized Bed coater (bottom-spray technology). A 95% portion of the IR beads is then coated by additional ER coating. The second Control Release coating is also performed in Wurster-equipped Fluidized Bed coater. Both portions are then mixed and encapsulated.

### Example 19: Tablet Dosage Form Containing IR and ER Memantine Components

The IR and ER memantine components of Example 18 are mixed with Polyethylene oxide (Polyox WSR-301), STARLAC™ and Magnesium stearate, and compressed into tablets.

### Example 20: Dissolution Procedure

| | |
|---|---|
| Equipment: | 6-vessel assembly, Apparatus I (Basket) |
| Medium: | 0.2% NaCl in 0.1N HCl |
| Volume: | 900 mL |
| Stirring Rate: | 100 RPM |
| Temperature: | 37° C. ± 0.5° C. |

What is claimed:

1. A modified release solid oral dosage form comprising
about 140 mg to about 200 mg of memantine, or a pharmaceutically acceptable salt thereof, contained in an immediate release component and an extended release component,
wherein the immediate release component comprises between about 5% and about 20% of the total amount of memantine, or the salt thereof, in the solid dosage form and provides for an immediate release of memantine, or the salt thereof;
the extended release component comprises between about 80% and about 95% of the total amount of memantine, or the salt thereof, in the solid dosage form and provides for an extended release of memantine, or the salt thereof, and
at least one pharmaceutically acceptable rate controlling excipient that comprises a mucoadhesive;
and wherein the solid dosage form provides a human in vivo plasma profile at steady state comprising
a Cₘₐₓ of about 100 ng/mL to about 160 ng/ml,
a Cₘᵢₙ of about 30 ng/ml to about 140 ng/mL, and
an AUCₜₐᵤ of more than about 14,000 ng h/ml; and
has a dissolution profile when measured using USP type 1, basket dissolution method at 100 rpm using 0.1 N HCl as the dissolution medium, pH 1.2, at 37° C.±0.5° C. of not more than 45% at 24 hours.

2. The modified release solid oral dosage form according to claim 1 adapted for administration once weekly to a patient in need thereof.

3. The modified release solid oral dosage form according to claim 1, wherein the dosage form comprises about 160 mg to about 200 mg of the memantine or the pharmaceutically acceptable salt thereof.

4. The modified release solid oral dosage form according to claim 1, wherein the dosage form comprises about 140 mg to about 190 mg of the memantine or the pharmaceutically acceptable salt thereof.

5. The modified release solid oral dosage form according to claim 1, wherein the dosage form comprises about 160 mg to about 190 mg of the memantine or the pharmaceutically acceptable salt thereof.

6. The modified release solid oral dosage form according to claim 1, wherein the dosage form comprises about 170 mg to about 200 mg of the memantine or the pharmaceutically acceptable salt thereof.

7. The modified release solid oral dosage form according to claim 1, wherein the dosage form comprises about 170 mg to about 190 mg of the memantine or the pharmaceutically acceptable salt thereof.

8. The modified release solid oral dosage form according to claim 1, wherein the dosage form comprises about 190 mg to about 200 mg of the memantine or the pharmaceutically acceptable salt thereof.

9. The modified release solid oral dosage form of claim 1 having the following in vivo plasma profile at steady state:
a Cₘₐₓ from about 100 ng/ml to about 140 ng/ml or from about 100 ng/ml to about 130 ng/ml;
a Cₘᵢₙ from about 30 ng/ml to about 125 ng/ml or from about 40 ng/ml to about 125 ng/ml or from about 50 ng/ml to about 125 ng/ml; and
an AUCₜₐᵤ from about 14,000 ng h/ml to about 25,000 ng h/ml or from about 15,000 ng h/ml to about 25,000 ng h/ml or from about 16,000 ng h/ml to about 25,000 ng h/ml or from about 17,000 ng h/ml to about 25,000 ng h/ml.

10. The modified release solid oral dosage form of claim 1 having the following in vivo plasma profile at steady state:
a Cₘₐₓ from 100 ng/ml to about 130 ng/ml;
a Cₘᵢₙ from about 50 ng/ml to about 125 ng/ml; and
an AUCₜₐᵤ from about 17,000 ng h/ml to about 25,000 ng h/ml.

11. The modified release solid oral dosage form according to claim 1, wherein the Cₘₐₓ is about 100 ng/ml to about 145 ng/ml.

12. The modified release solid oral dosage form according to claim 1, wherein the Cₘₐₓ is about 100 ng/ml to about 125 ng/ml.

13. The modified release solid oral dosage form according to claim 1, wherein Cₘᵢₙ is about 40 ng/ml to about 125 ng/ml or about 50 ng/ml to about 125 ng/ml.

14. The modified release solid oral dosage form according to claim 1, wherein the AUCₜₐᵤ is about 14,000 ng h/ml to about 25,000 ng h/ml.

15. The modified release solid oral dosage form according to claim 1, wherein the AUCₜₐᵤ is 15,000 ng h/ml or more.

16. The modified release solid oral dosage form according to claim 1, wherein the AUCₜₐᵤ is about 15,000 ng h/ml to about 25,000 ng h/ml.

17. The modified release solid oral dosage form according to claim 1, wherein the AUCₜₐᵤ is about 16,000 ng h/ml to about 25,000 ng h/ml.

18. The modified release solid oral dosage form according to claim 1, wherein the AUCₜₐᵤ is more than about 16,000 ng h/ml.

19. The modified release solid oral dosage form according to claim 1, wherein the dissolution rate is not more than 70% at 48 hours.

20. The modified release solid oral dosage form according to claim 1, wherein the dissolution profile is not more than 70% at 50 hours and more than about 80% after about 96 hours.

21. The modified release solid oral dosage form of claim 1, wherein the dissolution profile is not more than 35% at 24 hours.

22. The modified release solid oral dosage form of claim 1, wherein the dissolution profile is not more than 80% at 55 hours.

23. The modified release solid oral dosage form according to claim 1, wherein the dissolution profile is not more than 70% at 72 hours.

24. The modified release solid oral dosage form according to claim 1, wherein the dissolution profile is not more than about 80% after about 96 hours.

25. The modified release solid oral dosage form according to claim 1, wherein the in vivo plasma profile at steady state is further characterized by a memantine Tₘₐₓ of at least about 36 hours.

26. The modified release solid oral dosage form according to claim 1, wherein the in vivo plasma profile at steady state is further characterized by a memantine Tₘₐₓ of about 36 to 96 hours.

27. The modified release solid oral dosage form according to claim 1, wherein the in vivo plasma profile at steady state is further characterized by a memantine Tₘₐₓ of about 48 to 72 hours.

28. The modified release solid oral dosage form according to claim 1, wherein the pharmaceutically acceptable salt of memantine is hydrochloride salt or sulfate salt.

29. The modified release solid oral dosage form according to claim 1, wherein memantine is in the form of the hydrochloride salt.

30. The modified release solid oral dosage form according to claim 1, wherein the immediate release component is in the form of beads and the extended release component is in the form of beads coated with the at least one pharmaceutically acceptable rate controlling excipient.

31. The modified release solid oral dosage form according to claim 30, wherein the immediate release component comprises a core formed from memantine or a pharmaceutically acceptable salt thereof, and optionally a filler.

32. The modified release solid oral dosage form according to claim 31, wherein the filler is microcrystalline cellulose, lactose, sorbitol, dextrose, sucrose, mannitol, dibasic calcium phosphate, starch, or a mixture thereof.

33. The modified release solid oral dosage form according to claim 31, wherein the filler is microcrystalline cellulose.

34. The modified release solid oral dosage form according to claim 1, wherein at least 90% by weight of the memantine or a pharmaceutically acceptable salt thereof is in the extended release component.

35. The modified release solid oral dosage form according to claim 1, wherein 95% by weight of the memantine or a pharmaceutically acceptable salt thereof is in the extended release component.

36. The modified release solid oral dosage form according to claim 1, wherein the at least one pharmaceutically acceptable rate controlling excipient comprises a polymeric material constituting or in addition to said mucoadhesive.

37. The modified release solid oral dosage form according to claim 36, wherein the polymeric material is polyethylene oxide, ethyl cellulose, hydroxypropyl methylcellulose, polyvinyl alcohol, polyacrylates, polymethacrylates, ethyl acrylate-methyl methacrylate copolymers, hydroxypropyl cellulose, or a mixture thereof.

38. The modified release solid oral dosage form according to claim 36, further comprising a plasticizer.

39. The modified release solid oral dosage form according to claim 38, wherein the plasticizer is polyethylene glycol, triethyl citrate, tributyl citrate, glycerin, dibutyl sebacate, triacetin, diethylphthalate, or a mixture thereof.

40. The modified release solid oral dosage form according to claim 38, wherein the plasticizer is triethyl citrate.

41. The modified release solid oral dosage form according to claim 38, wherein the weight ratio of the plasticizer to the at least one pharmaceutically acceptable rate controlling excipient is from about 1:2 to about 1:10 in the extended release component.

42. The modified release solid oral dosage form according to claim 1, wherein the at least one pharmaceutically acceptable rate controlling excipient comprises two polymeric materials of which one is the mucoadhesive, and hydroxylpropylmethyl cellulose.

43. The modified release solid oral dosage form according to claim 1, wherein the total amount of the at least one pharmaceutically acceptable rate controlling excipient to the total weight of the dosage form is from about 8% to about 60%.

44. The modified release solid oral dosage form according to claim 1, wherein the total amount of the at least one pharmaceutically acceptable rate controlling excipient to the total weight of the dosage form is from about 8% to about 20%.

45. The modified release solid oral dosage form according to claim 1, wherein the total amount of the at least one pharmaceutically acceptable rate controlling excipient to the total weight of the dosage form is from about 50% to about 60%.

46. The modified release solid oral dosage form according to claim 1, wherein the total amount of the at least one pharmaceutically acceptable rate controlling excipient to the total weight of the dosage form is from about 19% to about 40%.

47. The modified release solid oral dosage form according to claim 1, wherein the total amount of the at least one pharmaceutically acceptable rate controlling excipient to the total weight of the dosage form is from about 30% to about 60%.

48. The modified release solid oral dosage form according to claim 1, wherein the amount of the at least one pharmaceutically acceptable rate controlling excipient is about 10 to about 50 wt % of the extended release component.

49. The modified release solid oral dosage form according to claim 1, wherein the amount of memantine or a pharmaceutically acceptable salt of memantine in the extended release component is about 10 to about 60 wt % relative to the weight of the extended release component.

50. The modified release solid oral dosage form according to claim 1 wherein the weight ratio of the at least one pharmaceutically acceptable rate controlling excipient to memantine or the pharmaceutically acceptable salt thereof is from about 1:0.2 to about 1:5.0 in the extended release component.

51. The modified release solid oral dosage form according to claim 1, further comprising a lubricant that is sodium stearyl fumarate, stearic acid, magnesium stearate, calcium stearate, zinc stearate, talc, glyceryl behenate, or a mixture thereof.

52. The modified release solid oral dosage form according to claim 51 wherein the lubricant is magnesium stearate.

53. The modified release solid oral dosage form according to claim 1, wherein the mucoadhesive is selected from the group consisting of water soluble or water insoluble hydrophilic polymers, polymers that have swellable networks, hydrogels, and polymers with groups that can cross-link with other polymers or with a mucous membrane.

54. The modified release solid oral dosage form according to claim 53, wherein the mucoadhesive is polyethylene oxide.

55. The modified release solid oral dosage form according to claim 1, wherein the mucoadhesive is present in an amount of about 5 to about 60 wt %.

56. The modified release solid oral dosage form according to claim 1, wherein the mucoadhesive is present in an amount of about 5 to about 20 wt % of the total weight of the dosage form.

57. The modified release solid oral dosage form according to claim 1, wherein the weight ratio of the memantine or the pharmaceutically acceptable salt thereof to the mucoadhesive is from about 1:2 to about 1:4.

58. The modified release solid oral dosage form according to claim 1, wherein the memantine or pharmaceutically acceptable salt and the mucoadhesive agent are provided in a matrix.

59. The modified solid oral dosage form according to claim 58, wherein the amount of mucoadhesive in the dosage form is from about 20 to about 80 wt %.

60. The modified solid oral dosage form according to claim 58, wherein the amount of mucoadhesive in the dosage form is from about 30 to about 70 wt %.

61. The modified solid oral dosage form according to claim 58, wherein the amount of memantine or pharmaceutically acceptable salt thereof in the dosage form is from about 5 to about 40 wt %.

62. The modified solid oral dosage form according to claim 58, wherein the weight ratio of the memantine or pharmaceutically acceptable salt thereof to mucoadhesive in the dosage form is from about 1:1 to about 1:10.

63. The modified solid oral dosage form according to claim 58, further comprising at least one filler, glidant, lubricant, or base.

64. The modified solid oral dosage form according to claim 63, wherein the filler is microcrystalline cellulose, lactose, sorbitol, dextrose, sucrose, mannitol, dibasic calcium phosphate, starch, or a mixture thereof.

65. The modified solid oral dosage form according to claim 63, wherein the filler is microcrystalline cellulose or lactose, or a mixture thereof.

66. The modified solid oral dosage form according to claim 63, wherein the glidant is colloidal silicon dioxide, magnesium stearate, talc, sodium stearyl fumarate, magnesium carbonate, starch, or a mixture thereof.

67. The modified solid oral dosage form according to claim 63, wherein the glidant is colloidal silicon dioxide.

68. The modified solid oral dosage form according to claim 63, wherein the lubricant is sodium stearyl fumarate, stearic acid, magnesium stearate, calcium stearate, zinc stearate, talc, glyceryl behenate, or a mixture thereof.

69. The modified solid oral dosage form according to claim 63, wherein the lubricant is magnesium stearate.

70. The modified solid oral dosage form according to claim 63, wherein the base is sodium carbonate.

71. The modified release solid oral dosage form according to claim 1 in the form of a one unit dosage form.

72. The modified release solid oral dosage form according to claim 1 in the form of a capsule.

73. The modified release solid oral dosage form according to claim 72, wherein the capsule size is -00- or smaller.

74. The modified release solid oral dosage form according to claim 1 in the form of a compressed tablet.

75. The modified release solid oral dosage form according to claim 1 in the form of a monolithic tablet.

76. The modified release solid oral dosage form according to claim 1 wherein said memantine or pharmaceutically acceptable salt thereof represents the only active ingredient in said dosage form.
